(19) United States
(12) Patent Application Publication
(10) Pub. No.: US 2017/0143764 A1
(43) Pub. Date: May 25, 2017

(54) MENSTRUAL BLOOD-DERIVED STEM CELLS FOR THE TREATMENT OF HUMAN PANCREATIC CARCINOMA

(71) Applicant: CELLS FOR CELLS S.A., Las Condes, Santiago (CL)

(72) Inventors: Francisca ALCAYAGA, Las Condes, Santiago (CL); Maroun KHOURY, Las Condes, Santiago (CL)

(21) Appl. No.: 15/319,141

(22) PCT Filed: Jun. 17, 2015
(86) PCT No.: PCT/IB2015/054571

§ 371 Date: Dec. 15, 2016

Related U.S. Application Data
(60) Provisional application No. 62/013,294, filed on Jun. 17, 2014.

(51) Int. Cl. A61K 35/14

(52) U.S. Cl. CPC A61K 35/14; A61K 2035/124

(57) ABSTRACT

A method and compound product are created from menstrual blood-derived mesenchymal stem cells (MenSCs) with an anti-tumor effect. In particular, the method and compound show the potential anti-tumor effect MenSCs can have on human pancreatic carcinoma, which has been analyzed on the Mia PaCa 2 human pancreatic carcinoma cell line (ATCC #CRL 1420). MenSCs have been proven to have an in vitro anti-tumor effect both in bi-dimensional cultures (monolayer) and in three-dimensional cultures (tumorspheres). Additionally, MenSCs slow down the appearance of pancreatic tumors when they are co-implanted with the pancreatic carcinoma cell line. MenSCs also have an in vivo therapeutic advantage in the treatment of human pancreatic carcinoma via intra-tumor injections.

### FIELD OF INVENTION

This invention is related to pharmaceutical formulations or compositions with anti-tumor effects comprised of menstrual blood-derived mesenchymal stem cells (MenSCs) used in the treatment of human carcinoma.

In particular, compositions according to this invention are used to prepare medicinal products for the treatment of pancreatic cancer.

The invention provides a method to obtain menstrual blood-derived cells, the cells obtained, their conditioned medium, their use for the treatment of pancreatic carcinoma, and the methods and/or compositions they are comprised of.

### BACKGROUND

Pancreatic cancer is the fourth cause of death in Western countries. Although progress has been made in the biology of the tumor and the development of various therapeutic approaches, the prognosis of pancreatic cancer is still poor, with a survival rate of less than 5% after 5 years. The poor prognosis of pancreatic cancer is mainly due to the malignant behavior of this disease. A high rate of local invasion and quick systemic dissemination are the ‘signs’ of pancreatic cancer. Furthermore, pancreatic cancer is resistant to most well-known cytotoxic drugs, which added to the high relapse rate after surgery results in a major problem for modern medicine.

Mesenchymal stem cells (MenSCs) have been investigated for cancer treatments because of their ability to go directly to the tumor and incorporate into stromal cells, although earlier research show contradictory results. Some publications mention that MSCs inhibit tumor growth, while others say they promote tumor growth and metastases. Additionally, depending on the type of MSCs or tumor cell, MSCs anti-tumor benefits are highly variable. Thus, various studies show that MSCs, including adult MSCs and those derived from placental tissue, have different effects on neoplastic cells (Klopp, A H. et al. Concise Review: Dissecting a discrepancy in the literature: Do Mesenchymal Stem Cells Support or Suppress Tumor Growth?. Stem Cells 29:11-19, 2011). Based on the foregoing we can deduce that the effect each mesenchymal cell origin has must be evaluated specifically on a particular type of tumor, and the behavior, effectiveness, and use of each cell type cannot be predicted a priori.

For example, the anti-tumor effect bone marrow-derived mesenchymal stem cells (BM-MSCs) have on a Panc 1 human pancreatic carcinoma cell line (ATCC #CRL 1469) has been previously described. However, BM-MSCs have a low availability and a low rate of proliferation, which is a time limiting factor when transferring the therapy to the clinic. Also, BM-MSCs cannot be obtained, isolated, and cultured in a simple and and periodical manner. Thus, a desirable feature in anti-tumor therapy with MSCs is that the source of origin of these cells allows their easy and quick isolation and proliferation.

### STATE OF THE ART

From the documents available as regards technique, patent WO2011087299 presents a pharmaceutical composition for the treatment of cancer, containing an extracellular matrix (ECM) of mesenchymal stem cells derived from umbilical cord blood as an active ingredient, where the ECM is obtained by a culture of said stem cells, from which the conditioned medium is then separated, and only the cells are removed. These types of mesenchymal cells, similarly to BM-MSCs, have a low availability and cannot be obtained periodically, apart from the fact that a preferred outcome is not revealed for a specific type of cancer and the action mechanism depends on the effect of the extracellular matrix to suppress the expression of certain cell markers (p-AKT o p-mTOR).

Also, patent KR100874613 (B1) presents a composition against various types of cancer, such as melanoma, as it inhibits the growth of cancer cells that comprise the solution of a culture of adipose tissue-derived stem cells obtained from its culture in a DMEM/F12 medium for 1 day or longer, which are then filtered. This type of composition, like the previous document, also comprises a conditioned medium and therefore, its effect depends directly on the factors and the combination of its components, which are not analyzed in detail nor is the specific action mechanism.

These documents differ from this invention as regards the source and cell dose used, which consequently will secrete different factors or eventually, if dealing with the same factors, will present different levels.

### BRIEF DESCRIPTION OF THE FIGURES

FIG. 1. Co-culture of Mia PaCa 2 cancer cells with MenSCs at a 2:1 ratio. The inhibition of tumor colony formation in regarding the single blood culture of Mia PaCa 2 cancer cells. (a) Representative images of CFU formation. (b) Statistical analysis of CFU formation. *p≦0.05.

FIG. 2. Co-culture of Mia PaCa 2 cancer cells with MenSCs at a 2:1 ratio. The pane on the right shows representative images at different times of the single three-dimensional MSCs cultures. The pane on the right shows representative images of the tumorspheres formed in the co-culture at different times. The images are representative of at least 3 independent experiments.

FIG. 3. Assessment of the anti-proliferative effect of MenSCs on Mia PaCa 2 pancreatic cancer cells by means of the test using the WST-1 assay. After 72 hours of co-culture of neoplastic cells together with mesenchymal cells at a 2:1 ratio, the activity of mitochondrial dehydrogenase using WST-1 was determined.

FIG. 4. Assessment of the cell invasion using a Transwell System (8 um pore size), seeding 150,000 Mia PaCA 2 in the upper chamber in the MenSCs conditioned medium. Migration is analyzed after 24 hours of incubation, where the cells are stained with crystal violet and quantified using Image J software. (a) Statistical analysis of cancer cell migration. (b) Representative images of tumor migration.

FIG. 5. Co-culture of Mia Paca2 pancreatic carcinoma cancer cells with MSCs at a 2:1 concentration ratio (neo: MSCs) in direct contact or separated by a transwell-type semipermeable membrane. After 72 hours in culture, the cells were stained with CD90-PeCγ7. (a) Representative image of the FACS evaluation of neoplastic cell apoptosis induced by MSCs; (b) Statistical analysis of the apoptosis evaluation; and (c) Apoptosis evaluation in the absence of cell contact. The data are expressed as the mean±SEM corresponding to 3 independent experiments.

FIG. 6. MSCs and/or MiaPaca2 pancreatic cancer cells were co-implanted at a 2:1 ratio (neo: MSCs ratio is 10×10⁶cell: 5×10⁶cell) subcutaneously into both flanks of the mouse (Control group or MiaPaca2, N=10; Group Miapaca2+BM-MSCs, N=10; Group MiaPaca2+MenSCs, N=10). (a) MenSCs' effect on the incidence of appearance of cancer. (b-d) MenSCs interaction with pancreatic carcinoma cells, where (b) shows the kinetics of tumor growth, (c) tumor weight, and (d) tumor size for each group assessed. (e-f) In vivo tumor proliferation analysis, assessed by Ki67 immunohistochemistry. Representative images of a field of cancer for each group (e) and statistical analysis of the positive Ki67 cell count (f). The data are expressed as the mean±of at least 2 SEM independent experiments (*p≦0.05, **p≦0.01, ***p≦00.001; * as regards control, # as regards BM-MSCs).

FIG. 7. MenSCs' effect on pancreatic carcinoma growth. (a) Mean tumor volume; (b) Percentage of tumor growth compared to day one; (c) Tumor weight; (d) Tumor density; (e) Representative images of the tumors. *p≦0.05 compared to the Mia PaCa 2 control; **p≦00.01 compared to the Mia PaCa 2 control.

FIG. 8. Dkk3 secretion in MenSCs compared to other MSCs sources. (a) Quantitative analysis of Dkk3 secretion in MSCs SN. (b) Quantification of Dkk3 by means of the western blot test on MenSCs and BM-MSCs. Representative image of at least 3 experiments. The data is expressed as the mean±SEM. (*p≦0.05, **p≦0.01).

### DETAILED DESCRIPTION OF THE INVENTION

This invention supplies menstrual blood-derived stem cells for use in the treatment of pancreatic carcinoma and methods for obtaining them, their conditioned medium, and/or compositions they are comprised of.

To this day, the effect of MenSCs on human pancreatic cancer has not been described.

In particular, this invention shows the anti-tumor potential of MenSCs in human pancreatic carcinoma, analyzed on a Mia PaCa 2 human pancreatic carcinoma cell line.

One of the advantages of anti-tumor therapy with MSCs is that these cells allow quick and easy isolation and proliferation, in addition to the surprising anti-tumor effect in highly aggressive neoplastic cells, as in the case of pancreatic cancer.

While it is true that the population of MenSCs, like bone marrow-derived stem cells (BM-MSCs), show an in vitro and in vivo anti-tumor potential on the Mia PaCa 2 pancreatic carcinoma cell line, MenSCs showed an in vitro anti-tumor effect at a 2:1 ratio of cancer cells: mesenchymal stem cells, both in bi-dimensional cultures (monolayer) and in three-dimensional cultures (tumorspheres). The cell dose may be used at a cancer cells: MSCs ratio between 10:1 to 1:1, although it may vary depending on the requirements of each case.

In addition, MenSCs delays the appearance of pancreatic tumors when co-implanted at a 2:1 concentration. According to this invention, it can be observed that MenSCs grant an in vivo therapeutic benefit in the treatment of human pancreatic carcinoma via intra-tumor injections at a concentration of 1.5×10⁶ cells/tumor.

While it is not the only mechanism that explains it, it has been proposed that the anti-tumor activity of MenSCs is due to the fact that it has a higher expression and secretion of Dkk3 protein (a Wnt pathway antagonist), which suggests a unique anti-tumor potential via Dkk3.

Assessment of the In Vitro Anti-Tumor Effect of MenSCs on the Mia PaCa 2 Human Pancreatic Carcinoma Cell Line.

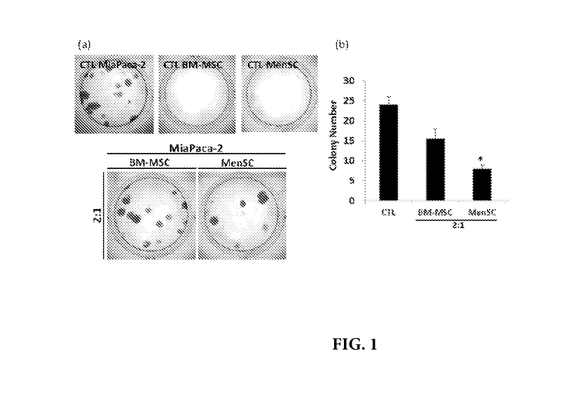

MenSC cells significantly inhibit the colony formation of pancreatic carcinoma cells, as seen in a bi-dimensional co-culture (monolayer) of Mia PaCa 2 cancer cells with MenScs at a 2:1 ratio compared to the results obtained in a single culture of Mia PaCa 2 cancer cells. FIG. 1(a) presents the determination of colony-forming units (CFU), where three single culture controls can be seen: Mia Paca 2, BM-MSC and MenSC versus the co-culture of Mia Paca 2 at a 2:1 ratio with BM-MSC and MenSC, respectively. The graph in FIG. 1(b) shows the statistical analysis of the formation of CFU, where a lower number of MenSCs co-culture colonies can be appreciated quantitatively, corresponding to about 50% or 33%, respectively, compared to the co-culture with BM-MSC or the Mia Paca 2 single culture.

Effect of the Size of Tumorspheres of Pancreatic Cancer Cells when Co-Cultured with MenSCs.

In order to determine the anti-tumor effect of MenSCs on tumorspheres, the cells were co-cultured at a cancer cells:MenSCs ratio of 2:1.

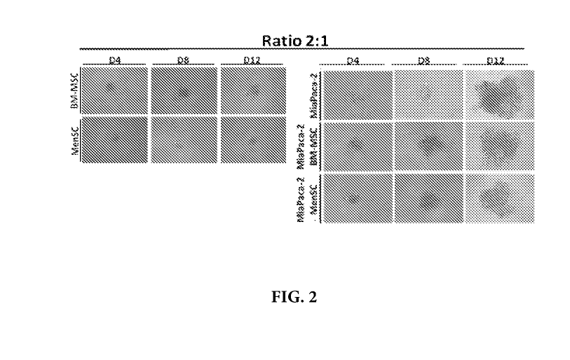

FIG. 2 shows that MenSCs inhibit the growth of tumorspheres of pancreatic cancer, compared to the single culture of Mia PaCa 2 cancer cells. The 6 figures in the left pane show representative images at different times of three-dimensional single MSCs cultures. The 9 figures in the right pane show representative images of the tumorspheres formed in the co-culture at different times. The images are representative of at least 3 independent experiments.

Effect of MenSCs in Cancer Cell Proliferation.

A proliferation test was conducted in order to assess the anti-tumor property of MenSCs on pancreatic carcinoma. In this particular case, a reagent test with WST-1 was used, which is a colorimetric method for the non-radioactive quantification of cell proliferation, viability, and cytotoxicity.

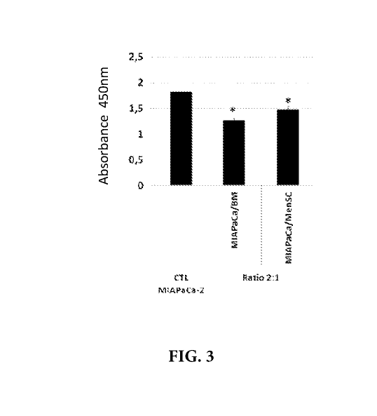

The activity of mitochondrial dehydrogenase was determined using WST-1 after 72 hours of co-culture of pancreatic neoplasms together with mesenchymal cells at a 2:1 ratio. The statistical analysis in FIG. 3 shows that both BM-MSCs and MenSCs are capable of inhibiting the proliferation of pancreatic cancer cells significantly, and no significant differences were observed between them.

Effect Determination of the Invasive Capacity of Cancer Cells in the Presence of MSCs

The invasive phenotype of neoplastic cells in the presence of the MenSCs conditioned medium was assessed.

The MenSC conditioned medium is obtained by centrifuging the MenSc culture and discarding the cells. The conditioned MenSC medium can be used directly, or concentrated or lyophilized.

The assessment was made using a Transwell System (8 um pore size), seeding 150,000 Mia PaCA 2 in the upper chamber in the MenSCs conditioned medium. After 24 hours of incubation, the cells that migrated to the lower chamber were stained with crystal violet and quantified using Image J software.

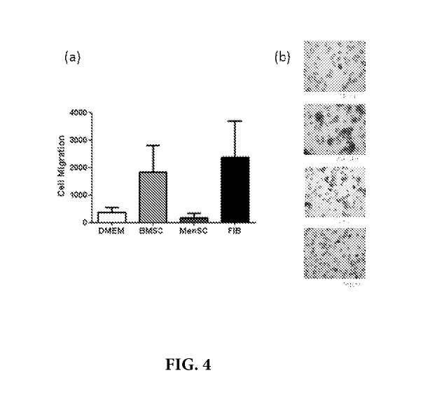

FIG. 4 shows that the MenSCs conditioned medium does not affect the invasive ability of pancreatic cancer cells, since there is no change in the invasive behavior. However, the BM-MSCs conditioned medium increased the invasive phenotype of cancer cells significantly, which proves that this type of cell would not be viable for the treatment of in vivo pancreatic cancer.

Assessment of Apoptosis Induction in Pancreatic Cancer Cells

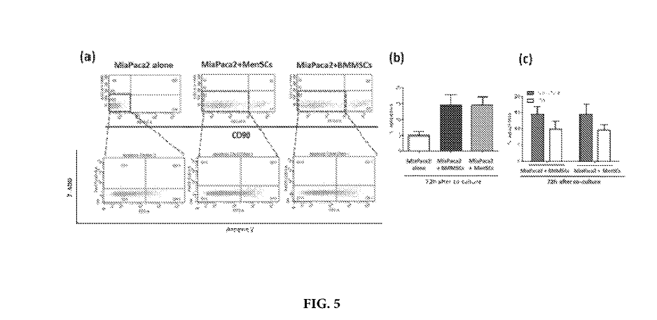

In the analysis, cancer cells from Mia Paca 2 pancreatic carcinoma we co-cultured at a 2:1 concentration (neo: MSCs) in direct contact or separated by a transwell-type semipermeable membrane. After 72 hours in culture, the cells were stained with CD90-PeCγ7 in order to differentiate the neoplastic cells (CD90−, population of the first lower quadrant of the graph, expanded) from MSCs (CD90+, population of the second lower quadrant), to subsequently assess the apoptosis exclusively in tumor cell populations with annexin V/7ADD staining with FACS (Fluorescence-Activated Cell Sorting) flow cytometry. FIG. 5(a) shows the assessment by FACS and FIGS. 5(b) and 5(c) show that there is apoptosis induction in the presence and absence of cell contact.

In conclusion, it was determined that there is apoptosis induction in neoplastic cells after the co-culture with BM-MSC and MenSCs in contact and in the absence of direct cell contact; consequently, apoptosis induction does not depend on cell-cell contact. It is important to consider that, while both cell types have a comparable in vitro effect, MenSCs cells have a more pronounced effect and, in in vivo tests, only the MenSCs show a beneficial effect.

In Vivo Determination of the Functional Consequence of Heterotopic Interaction between MenSCs and Pancreatic Carcinoma Cells (Onset)

The effect of MenSCs on tumor behavior after its implantation in a xenogeneic model was determined.

To do so, MSCs and/or Mia Paca 2 pancreatic cancer cells were co-implanted at a 2:1 ratio (neo: MSCs 10×10⁶ cell: 5×10⁶cell) subcutaneously into both flanks of the mouse (Control group or MiaPaca2, N=10; Group Miapaca2+BM-MSCs, N=10; Group MiaPaca2+MenSCs, N=10).

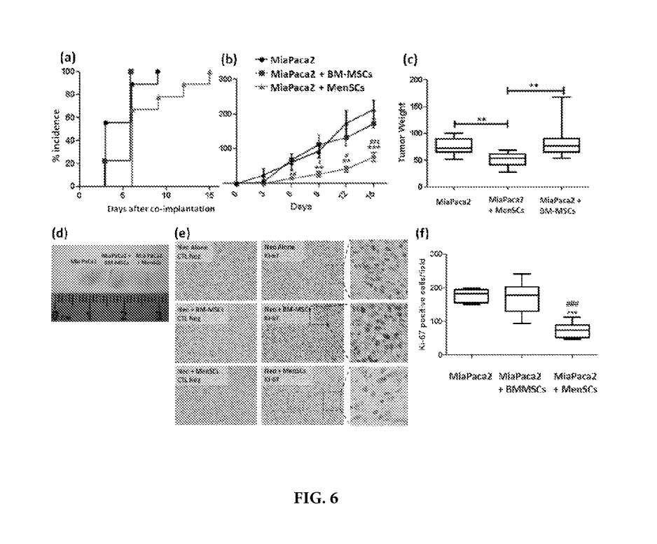

Each day, the area was examined to determine the date of appearance of the tumors, from which the size of the tumor was defined by measuring it with a caliper and the tumor volume was calculated using the 4/3πr³ formula. FIG. 6(a) shows that MenSCs have a significant impact on the incidence of appearance of cancer, delaying tumor onset for at least 3 days. FIGS. 6(b), 6(c) and 6(d) show that the interaction between MenSCs and pancreatic carcinoma cells heterotopically decreases tumor growth kinetics as early on as day 6 after co-implantation (FIG. 6(b)) and, consequently, tumor weight (FIG. 6(c)).

FIG. 6(d) shows a representative image of the tumor size for each group evaluated. FIGS. 6(e) and 6(f) show the analysis of in vivo tumor proliferation, evaluated using Ki67 immunohistochemistry, where a lower amount of cells with nuclear marking is observed in the group with MenSCs determined by counting 4 random fields of 4 tumors at 40× magnification.

In short, the co-transplant of pancreatic cancer cells with MenSCs significantly delays the appearance of tumors and therefore the kinetics of tumor growth compared to the control as well as to BM-MSCs. Likewise, cancer cell proliferation (revealed using IHC Ki67) shows a significant decrease of proliferation cells in vivo with MenSCs.

Thus, this invention provides a pharmaceutical composition for the treatment of pancreatic cancer and inhibiting pancreatic cancer cell proliferation, comprised of menstrual blood-derived mesenchymal stem cells (MenSCs), a fraction of said culture, or a combination of both.

In another embodiment of this invention, the composition comprises, optionally, an acceptable pharmaceutical vehicle.

### EXAMPLES

### Example 1

### Assessment of the Therapeutic Benefit provided by MenSCs on Pancreatic Carcinoma in a Xenograft Mouse Model

In vivo tests were conducted on NSG mice. The animals were implanted with Mia PaCa2 tumors. Reaching a tumor volume of 150 mm³ per group.

Then, the tumors were injected twice with MenSCs or BM-MSCs (1.5×10⁶ cells/tumor/iteration) on days 0 and 7. The size of the tumor was determined twice a week using a caliper and the tumor volume was calculated by using the 4/3πr³ formula.

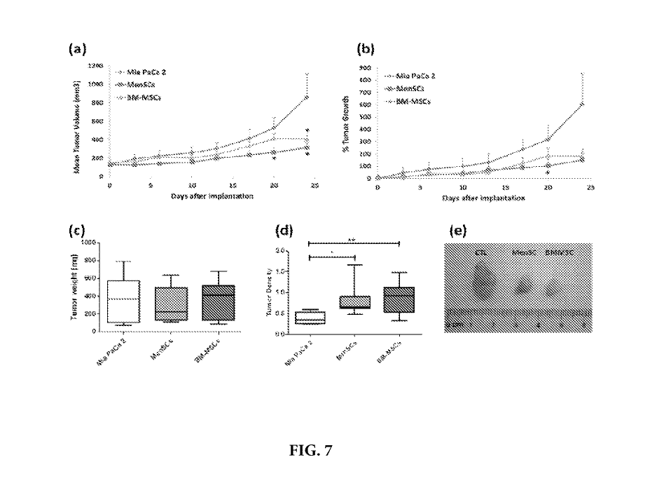

24 days after the first injection, the mice were put down and the tumors dissected. Tumor density was calculated by means of the d=m/v formula. FIG. 7(e) shows representative images of the tumors.

The graph in FIG. 7(a) shows the mean tumor volume, while the graph in FIG. 7(b) shows the percentage of tumor growth compared to day one. FIGS. 7(c) and 7(d) show the tumor weight and density, respectively. This suggests that MenSCs reduces the growth of in vivo Mia PaCa 2 pancreatic carcinoma tumors significantly. Furthermore, although the weight of the tumor at the end did not present differences between the groups, there was a significant increase in tumor density after treatment with MSCs.

### Example 2

### Determination of the Expression and Secretion of Dkk3 in Different Sources of Mesenchymal Stem Cells

In order to determine the expression and secretion of Dkk3, different types of mesenchymal cells were seeded at a concentration of 4×10⁵ cells in plates of 6-dishes in DMEM 2% FBS. The evaluated MSCs correspond to Chorion N=1; Decidua N=1, Umbilical Cord N=1, Dental Pulp N=1, Adipose Tissue N=1, Menstrual N=3 and Bone Marrow N=3.

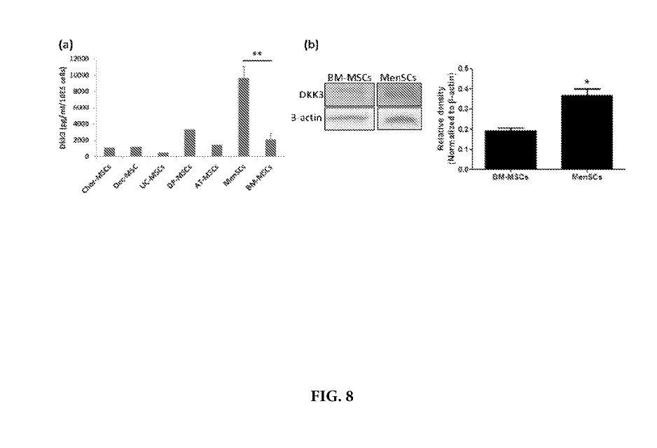

After 24 hours, the supernatant (SN) was collected and the cells counted. FIG. 8(a) shows the quantification of Dkk3 secretion in the MSCs supernatant by means of an ELISA analysis and relativized according to the number of cells per dish. Then, Dkk3 was quantified using the western blot test on MenSCs and BM-MSCs (FIG. 8(b)).

MenSCs proved to have the highest expression and secretion of the Dkk3 protein (Wnt pathway antagonist), which suggests a unique anti-tumor potential via Dkk3; FIG. 8(c) shows a representative image of at least 3 experiments.

1.-9. (canceled)

10. A method of treatment of pancreatic cancer which comprising: administering a pharmaceutical composition comprising menstrual blood-derived stem cells (MenSCs).

11. The method according to claim 11, wherein the pharmaceutical composition comprises MenSC stem cells and a pharmaceutically acceptable vehicle.

12. A method of treatment of pancreatic cancer comprising: administering a pharmaceutical composition comprising a MenSC conditioned medium.

13. The method according to claim 12, wherein the MenSC conditioned medium is obtained by centrifuging the MenSc culture and only discarding the cells.

14. The method according to claim 12, wherein the MenSC conditioned medium is concentrated or lyophilized.

15. The method according to claim 10, wherein a number of MenSCs is within a ratio between 10:1 to 1:1 of cancer cells: MSCs.

16. The method according to claim 10, wherein cancer cells in relation to MenSC cells administered have a ratio of 10×106 cells: 5×106 cells.
